(19) United States
(12) Patent Application Publication
(10) Pub. No.: US 2018/0280588 A1
(43) Pub. Date: Oct. 4, 2018

(54) IMPLANTABLE DEVICE WITH RECHARGEABLE BATTERY AND RECHARGE INTELLIGENCE

(71) Applicant: CARDIAC PACEMAKERS, INC., St. Paul, MN (US)

(72) Inventors: Keith R. Maile, New Brighton, MN (US); Jacob M. Ludwig, Isanti, MN (US); William J. Linder, Golden Valley, MN (US)

(73) Assignee: CARDIAC PACEMAKERS, INC., St. Paul, MN (US)

(21) Appl. No.: 15/940,395

(22) Filed: Mar. 29, 2018

Related U.S. Application Data
(60) Provisional application No. 62/480,766, filed on Apr. 3, 2017.

(51) Int. Cl. A61L 31/14; A61N 1/378; H02J 7/00

(52) U.S. Cl. CPC A61L 31/14; H02J 2007/0059; H02J 7/0052; A61N 1/3787

(57) ABSTRACT

An implantable medical device includes a rechargeable battery and a battery recharging assembly. The battery recharging assembly includes an energy receiver for capturing energy from an externally applied charging field, a battery charging circuit that is operably coupled to the rechargeable battery for recharging the rechargeable battery, and a demodulator that is operably coupled to the energy receiver and the battery charging circuit. The demodulator demodulates the energy captured by the energy receiver and delivers demodulated energy to the battery charging circuit to be used to charge the rechargeable battery. The IMD includes a controller that is configured to control operation of at least part of the IMD.

### CROSS REFERENCE TO RELATED APPLICATIONS

This application claims the benefit of U.S. Provisional Patent Application Ser. No. 62/480,766 filed on Apr. 3, 2017, the disclosure of which is incorporated herein by reference.

### TECHNICAL FIELD

The present disclosure pertains to medical devices, and more particularly to implantable medical devices that are capable of receiving power from another device

### BACKGROUND

Implantable medical devices are commonly used today to monitor physiological or other parameters of a patient and/or deliver therapy to a patient. In one example, to help patients with heart related conditions, various medical devices (e.g., pacemakers, defibrillators, etc.) can be implanted in a patient's body. Such devices may monitor and in some cases provide electrical stimulation (e.g. pacing, defibrillation, etc.) to the heart to help the heart operate in a more normal, efficient and/or safe manner. In another example, neuro stimulators can be used to stimulate tissue of a patient to help alleviate pain and/or other condition. In yet another example, an implantable medical device may simply be an implantable monitor that monitors one or more physiological or other parameters of the patient, and communicates the sensed parameters to another device such as another implanted medical device or an external device. In some cases, implantable medical devices may include rechargeable batteries that need to periodically be charged from a power source remote from the implantable medical devices.

### SUMMARY

The present disclosure pertains to medical devices, and more particularly to implantable medical devices that include rechargeable batteries. In one example, an implantable medical device (IMD) includes a housing and a rechargeable battery that is disposed within the housing. The IMD includes a battery recharging assembly that is disposed relative to the housing. The battery recharging assembly includes an energy receiver that is disposed relative to the housing for capturing energy from an externally applied charging field, a battery charging circuit that is disposed within the housing and operably coupled to the rechargeable battery for recharging the rechargeable battery, and a demodulator that is disposed within the housing and operably coupled to the energy receiver and the battery charging circuit. The demodulator demodulates the energy captured by the energy receiver and delivers demodulated energy to the battery charging circuit, the battery charging circuit using the demodulated energy to charge the rechargeable battery. The IMD includes a controller that is disposed within the housing and powered by the rechargeable battery, the controller configured to control operation of at least part of the IMD.

Alternatively or additionally, the battery recharging assembly may further include a switch that, when activated, is configured to prevent the battery charging circuit from recharging the rechargeable battery.

Alternatively or additionally, the battery recharging assembly may further include a switch that, when activated, is configured to switch off the energy receiver so that the energy receiver does not provide captured energy to the demodulator and thus the battery charging circuit.

Alternatively or additionally, the energy receiver may include a receiver coil having a first terminal and a second terminal, wherein the switch, when activated, effectively shorts the first terminal to the second terminal of the receiver coil.

Alternatively or additionally, the energy receiver may deliver a voltage to the demodulator, and the demodulator may be configured to step up the voltage delivered by the energy receiver and provide the stepped up voltage to the battery charging circuit.

Alternatively or additionally, the battery charging circuit may be configured to provide a constant current to the rechargeable battery while a power level of the rechargeable battery remains below a first threshold.

Alternatively or additionally, the battery charging circuit may be configured to provide a constant voltage to the rechargeable battery once the power level of the rechargeable battery exceeds the first threshold but remains below a second threshold.

Alternatively or additionally, the battery charging circuit may be configured to activate the switch when the power level of the rechargeable battery reaches the second threshold.

Alternatively or additionally, the energy receiver may include an inductive energy receiver and/or an RF energy receiver.

Alternatively or additionally, the energy receiver may include a receiver coil supported on the housing.

Alternatively or additionally, the receiver coil may include a trace disposed on the housing.

Alternatively or additionally, the IMD may further include a communications module that enables the controller to communicate with a remote device, and the controller may be configured to receive charging instructions from the remote device via the communications module.

Alternatively or additionally, the battery charging circuit may be configured to monitor a remaining power level within the rechargeable battery, and to determine when to activate the switch based at least in part on remaining power level within the rechargeable battery.

Alternatively or additionally, the energy receiver may be configured to receive energy for recharging the rechargeable battery from another IMD.

Alternatively or additionally, the IMD may be a leadless cardiac pacemaker (LCP) that includes a therapy module for delivering pacing therapy.

In another example, a leadless cardiac pacemaker (LCP) may be configured to sense cardiac signals from a patient's heart and to deliver pacing therapy to the patient's heart. The LCP includes a housing, a rechargeable battery disposed within the housing and a battery recharging assembly that is disposed relative to the housing. The battery recharging assembly includes an energy receiver that is disposed relative to the housing for capturing energy from an externally applied charging field, a battery charging circuit that is disposed within the housing and operably coupled to the rechargeable battery for recharging the rechargeable battery, and a demodulator that is disposed within the housing and operably coupled to the energy receiver and the battery charging circuit. The demodulator demodulates the energy captured by the energy receiver and delivers demodulated energy to the battery charging circuit, the battery charging circuit using the demodulated energy to charge the rechargeable battery. The battery recharging assembly includes a switch that, when activated, is configured to prevent the battery charging circuit from recharging the rechargeable battery. A pair of electrodes are secured relative to the housing. A controller is disposed within the housing and is powered by the rechargeable battery, the controller is configured to sense cardiac signals and/or to deliver pacing therapy via the pair of electrodes.

Alternatively or additionally, the battery charging circuit may be configured to monitor a remaining power level within the rechargeable battery, and to determine when to activate the switch.

Alternatively or additionally, the energy receiver may deliver a voltage to the demodulator, and the demodulator may be configured to step up the voltage delivered by the energy receiver and to provide the stepped up voltage to the battery charging circuit. The battery charging circuit may be configured to provide a constant current to the rechargeable battery while a power level of the rechargeable battery remains below a first threshold and to provide a constant voltage to the rechargeable battery once the power level of the rechargeable battery exceeds the first threshold but remains below a second threshold. The battery charging circuit may be configured to activate the switch when the power level of the rechargeable battery reaches the second threshold.

Alternatively or additionally, the switch, when activated, may be configured to switch off the energy receiver so that the energy receiver does not provide captured energy to the demodulator and thus the battery charging circuit.

In another example, a method of recharging an implantable device having a rechargeable battery includes subjecting the implantable device to an energy field so that the implantable device is able to receive energy from the energy field and allowing received energy to be used to recharge the rechargeable battery while a power level of the rechargeable battery is below a threshold. The method includes switching off received energy from being used to recharge the rechargeable battery when the power level of the rechargeable battery is at or above the threshold.

The above summary of some illustrative embodiments is not intended to describe each disclosed embodiment or every implementation of the present disclosure. The Figures, and Description, which follow, more particularly exemplify some of these embodiments.

### BRIEF DESCRIPTION OF THE DRAWINGS

The disclosure may be more completely understood in consideration of the following description in connection with the accompanying drawings, in which:

FIG. 1 is a schematic block diagram of a system that includes one or more implantable medical devices having rechargeable batteries in accordance with an example of the disclosure;

FIG. 2 is a schematic block diagram of an implantable medical device (IMD) useable in the system of FIG. 1;

FIG. 3 is a schematic block diagram of a battery recharging assembly usable within the IMD of FIG. 2;

FIG. 4 is a schematic block diagram of a battery recharging assembly usable within the IMD of FIG. 2;

FIG. 5 is a schematic block diagram of an implantable medical device (IMD) useable in the system of FIG. 1;

FIG. 6 is a schematic block diagram of a leadless cardiac pacemaker (LCP) useable in the system of FIG. 1;

FIG. 7 is a schematic view of an LCP in accordance with the disclosure;

FIG. 8 is a schematic view of an LCP in accordance with the disclosure;

FIG. 9 is a schematic view of an LCP in accordance with the disclosure;

FIG. 10 is a flow diagram illustrating a method that may be carried out using the system of FIG. 1;

FIG. 11 is a schematic block diagram of an illustrative IMD in accordance with an example of the disclosure; and

FIG. 12 is a schematic block diagram of another illustrative medical device that may be used in conjunction with the IMD of FIG. 11.

While the disclosure is amenable to various modifications and alternative forms, specifics thereof have been shown by way of example in the drawings and will be described in detail. It should be understood, however, that the intention is not to limit the disclosure to the particular embodiments described. On the contrary, the intention is to cover all modifications, equivalents, and alternatives falling within the spirit and scope of the disclosure.

### DESCRIPTION

For the following defined terms, these definitions shall be applied, unless a different definition is given in the claims or elsewhere in this specification.

All numeric values are herein assumed to be modified by the term “about,” whether or not explicitly indicated. The term “about” generally refers to a range of numbers that one of skill in the art would consider equivalent to the recited value (i.e., having the same function or result). In many instances, the terms “about” may include numbers that are rounded to the nearest significant figure.

The recitation of numerical ranges by endpoints includes all numbers within that range (e.g. 1 to 5 includes 1, 1.5, 2, 2.75, 3, 3.80, 4, and 5).

As used in this specification and the appended claims, the singular forms “a”, “an”, and “the” include plural referents unless the content clearly dictates otherwise. As used in this specification and the appended claims, the term “or” is generally employed in its sense including “and/or” unless the content clearly dictates otherwise.

It is noted that references in the specification to “an embodiment”, “some embodiments”, “other embodiments”, etc., indicate that the embodiment described may include one or more particular features, structures, and/or characteristics. However, such recitations do not necessarily mean that all embodiments include the particular features, structures, and/or characteristics. Additionally, when particular features, structures, and/or characteristics are described in connection with one embodiment, it should be understood that such features, structures, and/or characteristics may also be used connection with other embodiments whether or not explicitly described unless clearly stated to the contrary.

The following detailed description should be read with reference to the drawings in which similar structures in different drawings are numbered the same. The drawings, which are not necessarily to scale, depict illustrative embodiments and are not intended to limit the scope of the disclosure. While the present disclosure is applicable to any suitable implantable medical device (IMD), the description below often uses implantable cardioverter-defibrillator (ICD) and/or pacemakers as particular examples.

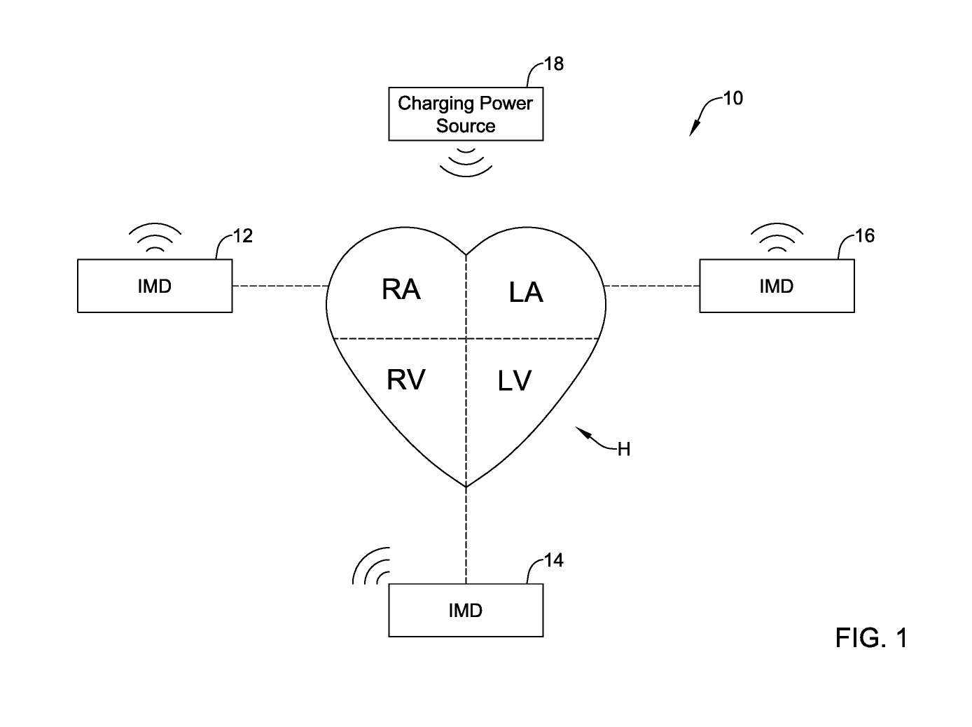

FIG. 1 is a schematic diagram showing an illustrative system 10 that may be used to sense and/or pace a heart H. In some cases, the system 10 may also be configured to shock the heart H. The heart H includes a right atrium RA and a right ventricle RV. The heart H also includes a left atrium LA and a left ventricle LV. In some cases, the system 10 may include one or more medical devices that provide anti-arrhythmic therapy to the heart H. For example, and as illustrated, the system 10 may include an implantable medical device (IMD) 12, an implantable medical device (IMD) 14 and an implantable medical device (IMD) 16. It will be appreciated that this is merely illustrative, as in some cases there may only be one or two implantable medical devices, or there may be four or more implantable medical devices. In some cases, each of the IMD 12, the IMD 14 and the IMD 16 may be implanted at differing locations within or near the heart H. One or more of the IMD 12, the IMD 14 and the IMD 16 may be configured to sense cardiac activity. In some cases, one or more of the IMD 12, the IMD 14 and the IMD 16 may be configured to also provide therapy such as but not limited to pacing therapy to the heart H.

In some cases, each of the IMD 12, the IMD 14 and the IMD 16 may include a power supply such as a rechargeable battery or a capacitor that provides power for operation of the IMD 12, the IMD 14 and the IMD 16 may require periodic charging or recharging in order to have sufficient power to continue to power operation. In some cases, for example, the system 10 may include a charging power source 18 that is configured to create an energy field within the patient that can be harnessed by the IMD 12, the IMD 14 and the IMD 16 and used to recharge their power supply. In some cases, the charging power source 18 may be an energy source configured to provide inductive or RF energy from a position exterior to the patient. For example, the charging power source 18 may be a handheld device that the patient may periodically hold up against their chest. In some cases, the charging power source 18 may be another implanted device such as an implantable cardioverter-defibrillator (ICD) or a subcutaneous implantable cardioverter-defibrillator (SICD).

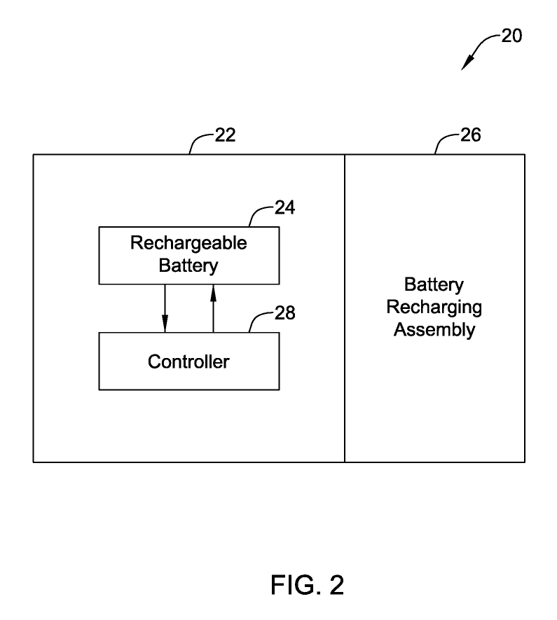

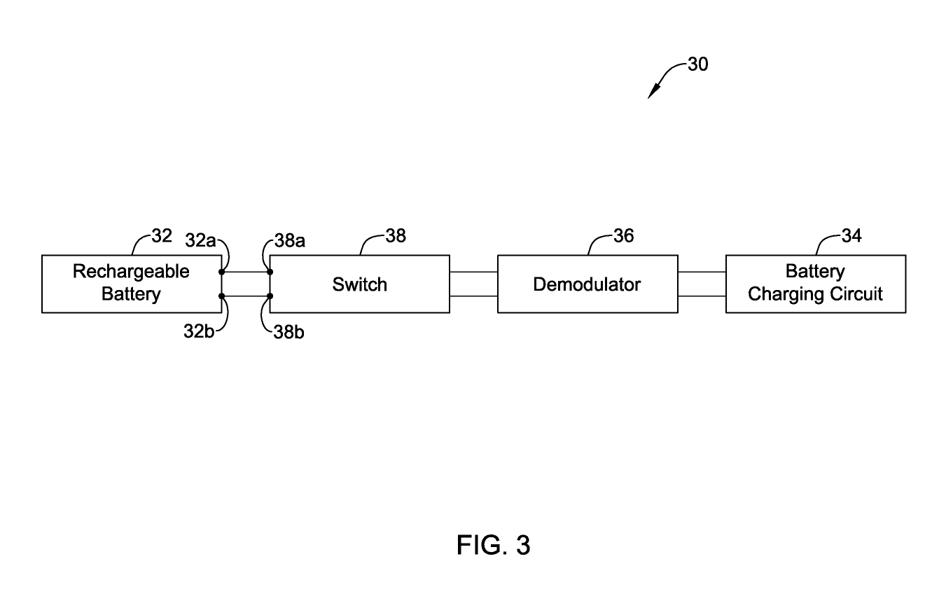

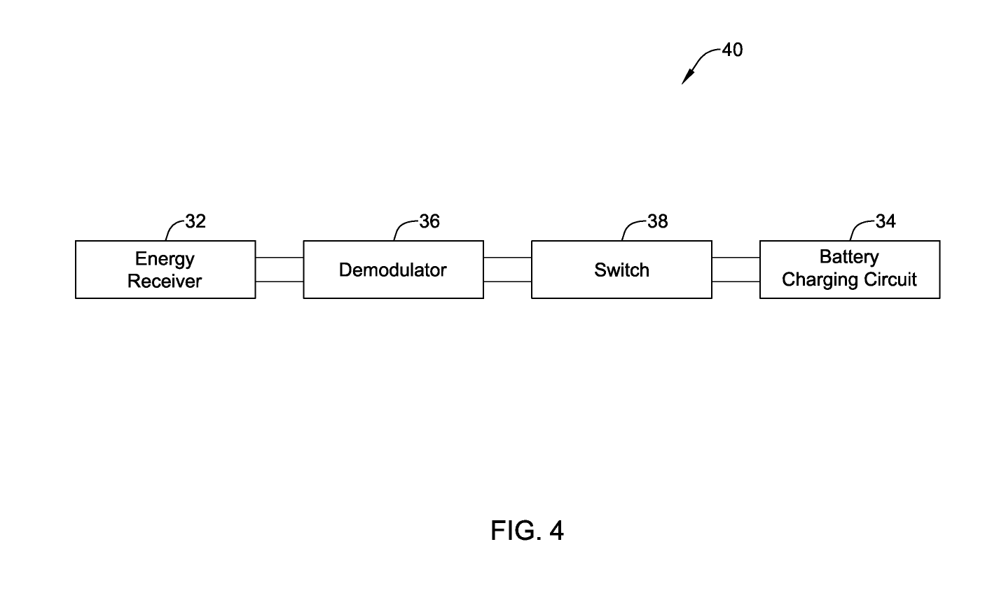

FIG. 2 is a schematic block diagram of an implantable medical device (IMD) 20 that may be considered as being an example of one of the IMD 12, the IMD 14 and the IMD 16 shown in FIG. 1. The IMD 20 includes a housing 22 and a rechargeable battery 24 that is disposed within the housing 22. A battery recharging assembly 26 is disposed relative to the housing 22. A controller 28 is disposed within the housing 22 and is powered by the rechargeable battery 24. In some cases, the controller 28 may be configured to control operation of at least part of the IMD 20. FIGS. 3 and 4 provide illustrative but non-limiting examples of battery recharging assemblies that may be used as the battery recharging assembly 26.

FIG. 3 is a schematic block diagram of a battery recharging assembly 30. The battery recharging assembly 30 includes an energy receiver 32 that may be considered as being disposed relative to the housing 22 (FIG. 2) for capturing energy from an externally applied charging field. The externally applied charging field may, for example, be provided by the charging power source 18 (FIG. 1). An energy receiver 32 may be disposed relative to the housing 22 (FIG. 2) for capturing energy from an externally applied charging field, such as may be applied via the charging power source 18 (FIG. 1). A battery charging circuit 34 may be disposed within the housing 22 and may be operably coupled to the rechargeable battery 24 for recharging the rechargeable battery 24. A demodulator 36 may be disposed within the housing 22 and may be operably coupled to the energy receiver 32 and the battery charging circuit 34. In some cases, the demodulator 36 demodulates the energy captured by the energy receiver 32 and delivers demodulated energy to the battery charging circuit 34 for the battery charging circuit 34 to use to charge the rechargeable battery.

In some cases, as shown in FIG. 3, the battery recharging assembly may further include a switch 38 that, when activated, may be configured to switch off the energy receiver 32 so that the energy receiver 32 does not provide captured energy to the demodulator 36 and thus does not provide captured energy to the battery charging circuit 34. In some cases, the energy receiver 32 may include a first terminal 32a and a second terminal 32b, and the switch 38 may include a first terminal 38a that is electrically connectable to the first terminal 32a and a second terminal 38b that is electrically connectable to the second terminal 32b such that the switch 38, when activated, effectively shorts the first terminal 32a to the second terminal 32b. In some cases, the switch 38 may effectively ground out the energy receiver 32.

FIG. 4 is a schematic block diagram of a battery recharging assembly 40 in which the switch 38 is operatively disposed between the demodulator 36 and the battery charging circuit 34. In some cases, the switch 38, when activated, may be configured to prevent the battery charging circuit 34 from recharging the rechargeable battery 24 by preventing power from getting to the battery charging circuit 34. In some cases, the switch 38 may be operably coupled between the battery charging circuit 34 and the rechargeable battery 24, to prevent the battery charging circuit 34 from charging the rechargeable battery 24.

In some cases, regardless of where the switch 38 may be located, the energy receiver 32 delivers a voltage to the demodulator 36, and the demodulator 36 may be configured to step up the voltage delivered by the energy receiver 32 and provide the stepped up voltage to the battery charging circuit 34. In some cases, the battery charging circuit 34 may be configured to provide a constant current to the rechargeable battery 24 while a power level of the rechargeable battery 24 remains below a first threshold and to provide a constant voltage to the rechargeable battery 24 once the power level of the rechargeable battery 24 exceeds the first threshold but remains below a second threshold. In some instances, the battery charging circuit 34 may be configured to activate the switch 38 when the power level of the rechargeable battery 24 reaches the second threshold. In some cases, the first threshold may represent a relative power level of about 85 to 95 percent, or in some cases a relative power level of about 90 percent. In some cases, the second threshold may represent a relative power level of at least about 95 percent, or in some cases a relative power level of about 100 percent.

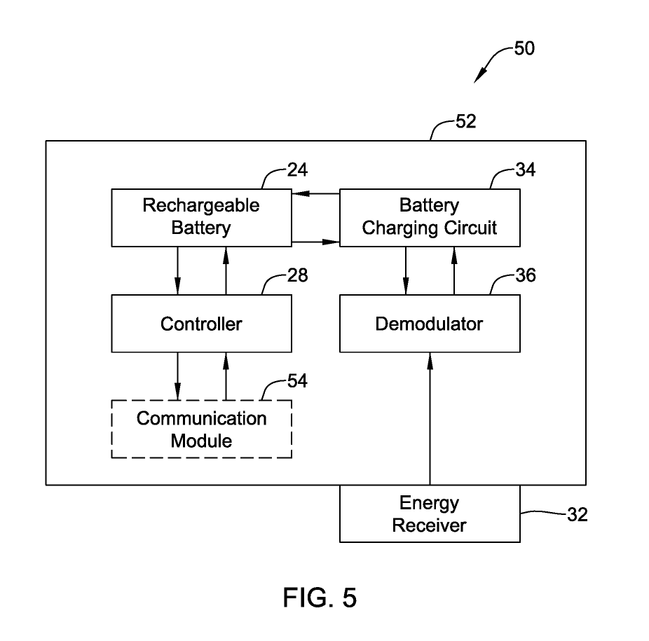

FIG. 5 is a schematic block diagram of an implantable medical device (IMD) 50 having a housing 52. The rechargeable battery 24 is operably coupled with the controller 28 and with the battery charging circuit 34. The energy receiver 32 is operably coupled with the demodulator 36, which in turn is operably coupled with the battery charging circuit 34. In some cases, the energy receiver 32 may be an inductive energy receiver and/or an RF energy receiver. In some cases, the energy receiver 32 may include a receiver coil that is supported on the housing 52. In some instances, the receiver coil may include a trace that is disposed on the housing 52.

In some cases, the IMD 50 may include a communications module 54 that is operably coupled with the controller 28 such that the controller 28 is able to communicate with a remote device, and the controller 28 may be able to receive charging instructions from the remote device via the communications module 54. In some cases, the remote device may be an ICD or SICD that is implanted within the patient but remote from the IMD 50. In some instances, the battery charging circuit 34 may be configured to monitor a remaining power level within the rechargeable battery 24, and to determine when to activate the switch 38 based at least in part on remaining power level within the rechargeable battery 24. In some cases, the energy receiver 32 may be configured to receive energy for recharging the rechargeable battery 24 from another IMD.

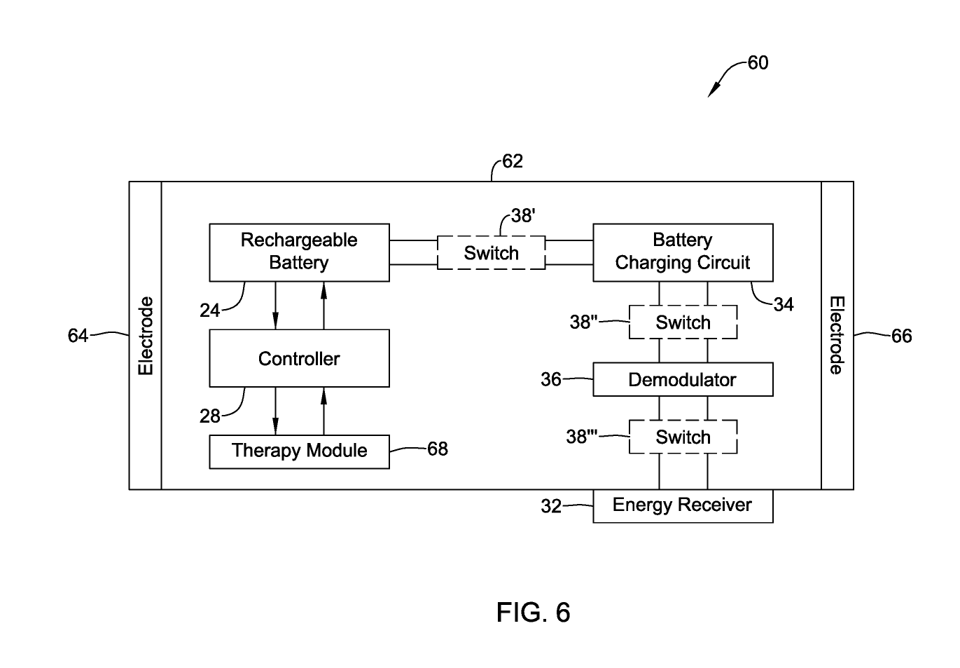

FIG. 6 is a schematic block diagram of a leadless cardiac pacemaker (LCP) 60 that is configured to sense cardiac signals from a patient's heart and to deliver pacing therapy to the patient's heart. The LCP 60 includes a housing 62 and the rechargeable battery 24 that is disposed within the housing 62. The LCP 60 includes the energy receiver 32 for capturing energy from an externally applied charging field and the battery charging circuit 34 operably coupled to the rechargeable battery 24. The demodulator 36 is operably coupled to the energy receiver 32 and the battery charging circuit 34 and demodulates the energy captured by the energy receiver 32 and delivers the demodulated energy to the battery charging circuit 34. The battery charging circuit 34 uses the demodulated energy to charge the rechargeable battery 24. The IMD 60 includes an electrode 64 and an electrode 66. While two electrodes 64, 66 are shown, in some cases the IMD 60 may include three or more electrodes. In some cases, the controller 28 may be operably coupled to, or may integrally include, a therapy module 68 that is configured to deliver pacing therapy via the electrodes 64, 66.

In some cases, the IMD 60 includes a switch to selectively prevent the battery charging circuit 34 from recharging the rechargeable battery 24. This may be beneficial, for example, if the rechargeable battery 24 is already fully charged, or if another implantable device has a more urgent need for power. The switch may be in any of a variety of locations, as indicated. One possible location is a switch 38′, operably located between the rechargeable battery 24 and the battery charging circuit 34. Another possible location is a switch 38″, operably located between the battery charging circuit 34 and the demodulator 36. Another possible location is a switch 38′″, operably located between the demodulator 36 and the energy receiver 32.

In some cases, the battery charging circuit 34 may be configured to monitor a remaining power level within the rechargeable battery 24, and to determine when to activate the switch. In some cases, the energy receiver 32 delivers a voltage to the demodulator 36, and the demodulator 36 may be configured to step up the voltage delivered by the energy receiver 32 and provide the stepped up voltage to the battery charging circuit 34. In some instances, the battery charging circuit 34 may be configured to provide a constant current to the rechargeable battery 24 while a power level of the rechargeable battery 24 remains below a first threshold and to provide a constant voltage to the rechargeable battery 24 once the power level of the rechargeable battery exceeds the first threshold but remains below a second threshold. The battery charging circuit 34 may be configured to activate the switch when the power level of the rechargeable battery 24 reaches the second threshold. The first threshold may be about 90 percent and the second threshold may be close to 100 percent, for example.

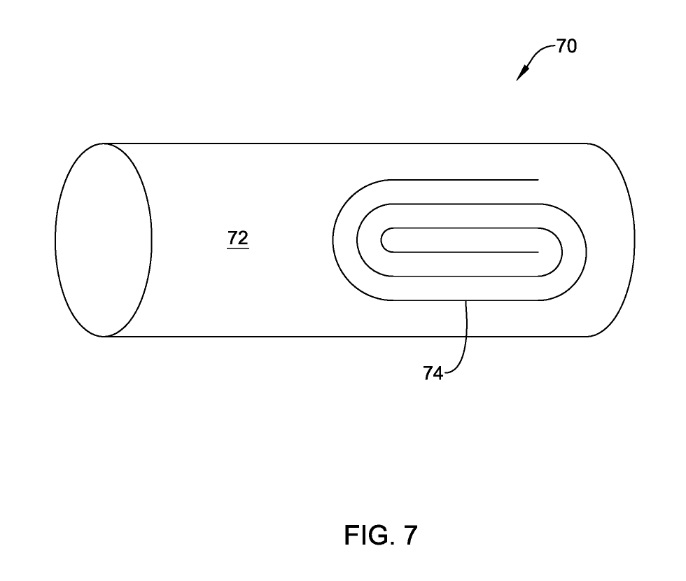

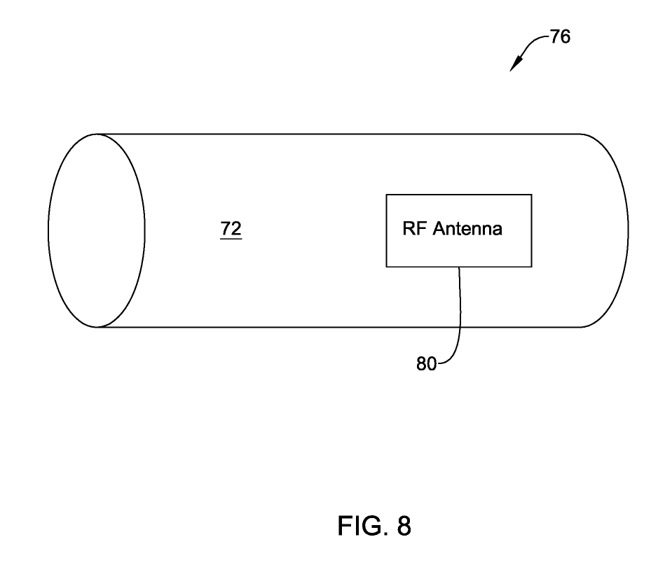

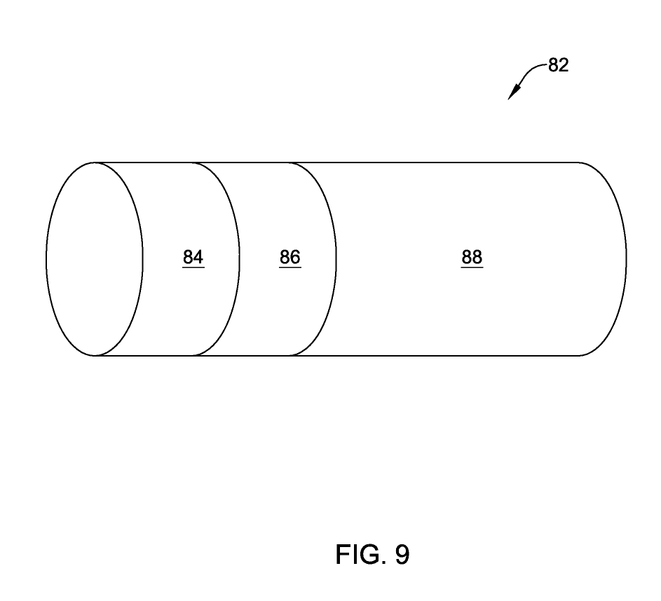

FIGS. 7 through 9 provides schematic illustrations of how energy receivers may be incorporated into an LCP. In FIG. 7, an LCP 70 has an outer housing surface 72, and a coil 74 that is formed on the outer housing surface 72. In some cases, the coil 74 may be a trace that is deposited or otherwise formed on the outer housing surface 72. In FIG. 8, an RF antenna 80 is disposed on an outer housing surface of an LCP 76. In some cases, the RF antenna 80 may be conformal. In FIG. 9, an LCP 82 includes a first section 84 that is devoted to an energy receiver, a second section 86 that is devoted to LCP electronics, and a third section 88 that is devoted to power storage. The LCP 82 may represent a length increase, but does not increase the diameter of the device.

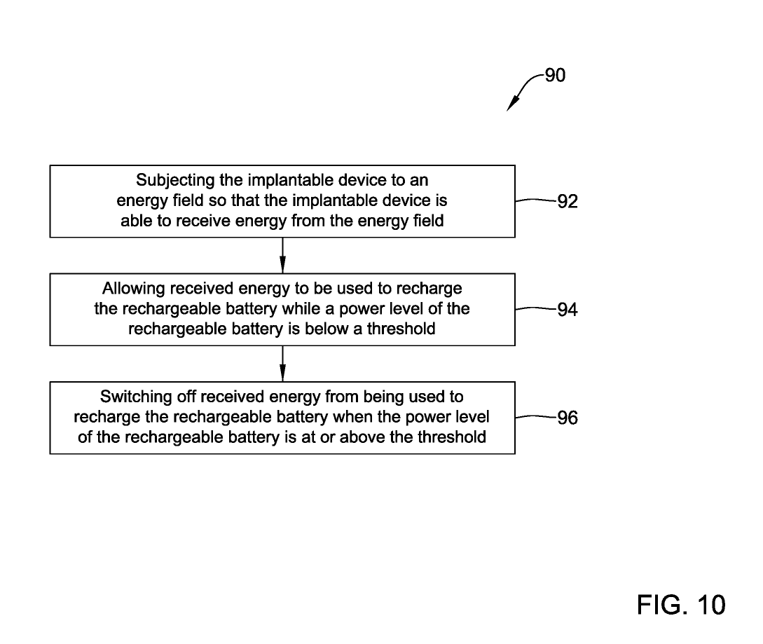

FIG. 10 is a flow diagram showing a method 90 of recharging an implantable device that has a rechargeable battery. The method includes subjecting the implantable device to an energy field so that the implantable device is able to receive energy from the energy field, as generally indicated at block 92. As can be seen at block 94, received energy is allowed to be used to recharge the rechargeable battery while a power level of the rechargeable battery is below a threshold. The received energy is switched off from being used to recharge the rechargeable battery when the power level of the rechargeable battery is at or above the threshold, as indicated at block 96.

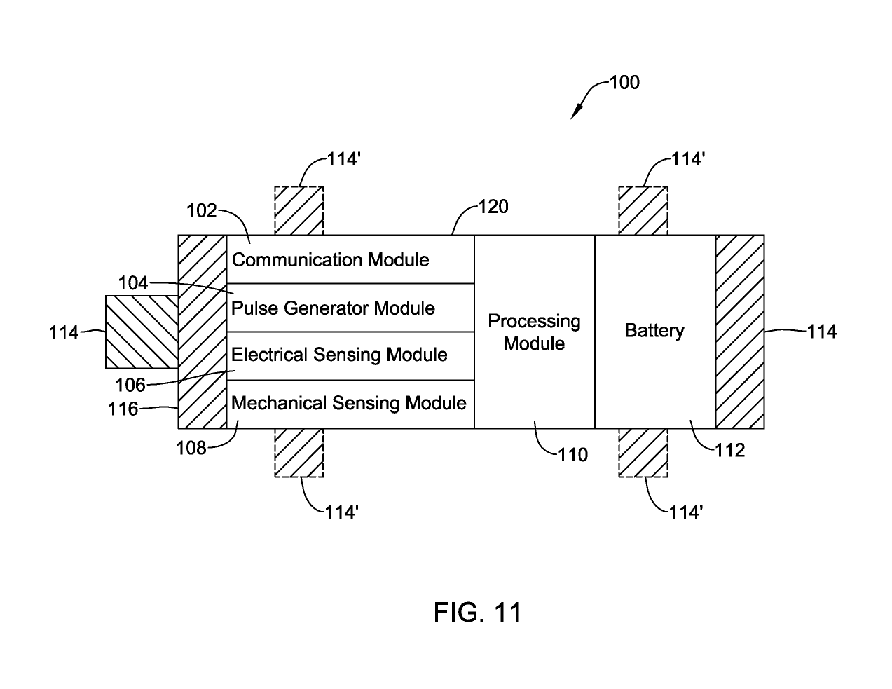

FIG. 11 depicts an illustrative leadless cardiac pacemaker (LCP) that may be implanted into a patient and may operate to deliver appropriate therapy to the heart, such as to deliver anti-tachycardia pacing (ATP) therapy, cardiac resynchronization therapy (CRT), bradycardia therapy, and/or the like. As can be seen in FIG. 11, the LCP 100 may be a compact device with all components housed within the or directly on a housing 120. In some cases, the LCP 100 may be considered as being an example of the IMD 12, the IMD 14, the IMD 16 (FIG. 1), the IMD 20 (FIG. 2), the IMD 50 (FIG. 5) or the LCP 60 (FIG. 6). In the example shown in FIG. 11, the LCP 100 may include a communication module 102, a pulse generator module 104, an electrical sensing module 106, a mechanical sensing module 108, a processing module 110, a battery 112, and an electrode arrangement 114. The LCP 100 may also include a receive coil for receiving inductive power, and a recharge circuit for recharging the battery 112 (or capacitor) using the received inductive power. It is contemplated that the LCP 100 may include more or fewer modules, depending on the application.

The communication module 102 may be configured to communicate with devices such as sensors, other medical devices such as an SICD, another LCP, and/or the like, that are located externally to the LCP 100. Such devices may be located either external or internal to the patient's body. Irrespective of the location, external devices (i.e. external to the LCP 100 but not necessarily external to the patient's body) can communicate with the LCP 100 via communication module 102 to accomplish one or more desired functions. For example, the LCP 100 may communicate information, such as sensed electrical signals, data, instructions, messages, R-wave detection markers, etc., to an external medical device (e.g. SICD and/or programmer) through the communication module 102. The external medical device may use the communicated signals, data, instructions, messages, R-wave detection markers, etc., to perform various functions, such as determining occurrences of arrhythmias, delivering electrical stimulation therapy, storing received data, and/or performing any other suitable function. The LCP 100 may additionally receive information such as signals, data, instructions and/or messages from the external medical device through the communication module 102, and the LCP 100 may use the received signals, data, instructions and/or messages to perform various functions, such as determining occurrences of arrhythmias, delivering electrical stimulation therapy, storing received data, and/or performing any other suitable function. The communication module 102 may be configured to use one or more methods for communicating with external devices. For example, the communication module 102 may communicate via radiofrequency (RF) signals, inductive coupling, optical signals, acoustic signals, conducted communication signals, and/or any other signals suitable for communication.

In the example shown in FIG. 8, the pulse generator module 104 may be electrically connected to the electrodes 114. In some examples, the LCP 100 may additionally include electrodes 114′. In such examples, the pulse generator 104 may also be electrically connected to the electrodes 114′. The pulse generator module 104 may be configured to generate electrical stimulation signals. For example, the pulse generator module 104 may generate and deliver electrical stimulation signals by using energy stored in the battery 112 within the LCP 100 and deliver the generated electrical stimulation signals via the electrodes 114 and/or 114′. Alternatively, or additionally, the pulse generator 104 may include one or more capacitors, and the pulse generator 104 may charge the one or more capacitors by drawing energy from the battery 112. The pulse generator 104 may then use the energy of the one or more capacitors to deliver the generated electrical stimulation signals via the electrodes 114 and/or 114′. In at least some examples, the pulse generator 104 of the LCP 100 may include switching circuitry to selectively connect one or more of the electrodes 114 and/or 114′ to the pulse generator 104 in order to select which of the electrodes 114/114′ (and/or other electrodes) the pulse generator 104 delivers the electrical stimulation therapy. The pulse generator module 104 may generate and deliver electrical stimulation signals with particular features or in particular sequences in order to provide one or multiple of a number of different stimulation therapies. For example, the pulse generator module 104 may be configured to generate electrical stimulation signals to provide electrical stimulation therapy to combat bradycardia, tachycardia, cardiac synchronization, bradycardia arrhythmias, tachycardia arrhythmias, fibrillation arrhythmias, cardiac synchronization arrhythmias and/or to produce any other suitable electrical stimulation therapy. Some more common electrical stimulation therapies include anti-tachycardia pacing (ATP) therapy, cardiac resynchronization therapy (CRT), and cardioversion/defibrillation therapy. In some cases, the pulse generator 104 may provide a controllable pulse energy. In some cases, the pulse generator 104 may allow the controller to control the pulse voltage, pulse width, pulse shape or morphology, and/or any other suitable pulse characteristic.

In some examples, the LCP 100 may include an electrical sensing module 106, and in some cases, a mechanical sensing module 108. The electrical sensing module 106 may be configured to sense the cardiac electrical activity of the heart. For example, the electrical sensing module 106 may be connected to the electrodes 114/114′, and the electrical sensing module 106 may be configured to receive cardiac electrical signals conducted through the electrodes 114/114′. The cardiac electrical signals may represent local information from the chamber in which the LCP 100 is implanted. For instance, if the LCP 100 is implanted within a ventricle of the heart (e.g. RV, LV), cardiac electrical signals sensed by the LCP 100 through the electrodes 114/114′ may represent ventricular cardiac electrical signals. In some cases, the LCP 100 may be configured to detect cardiac electrical signals from other chambers (e.g. far field), such as the P-wave from the atrium.

The mechanical sensing module 108 may include one or more sensors, such as an accelerometer, a pressure sensor, a heart sound sensor, a blood-oxygen sensor, a chemical sensor, a temperature sensor, a flow sensor and/or any other suitable sensors that are configured to measure one or more mechanical/chemical parameters of the patient. Both the electrical sensing module 106 and the mechanical sensing module 108 may be connected to a processing module 110, which may provide signals representative of the sensed mechanical parameters. Although described with respect to FIG. 11 as separate sensing modules, in some cases, the electrical sensing module 106 and the mechanical sensing module 108 may be combined into a single sensing module, as desired.

The electrodes 114/114′ can be secured relative to the housing 120 but exposed to the tissue and/or blood surrounding the LCP 100. In some cases, the electrodes 114 may be generally disposed on either end of the LCP 100 and may be in electrical communication with one or more of the modules 102, 104, 106, 108, and 110. The electrodes 114/114′ may be supported by the housing 120, although in some examples, the electrodes 114/114′ may be connected to the housing 120 through short connecting wires such that the electrodes 114/114′ are not directly secured relative to the housing 120. In examples where the LCP 100 includes one or more electrodes 114′, the electrodes 114′ may in some cases be disposed on the sides of the LCP 100, which may increase the number of electrodes by which the LCP 100 may sense cardiac electrical activity, deliver electrical stimulation and/or communicate with an external medical device. The electrodes 114/114′ can be made up of one or more biocompatible conductive materials such as various metals or alloys that are known to be safe for implantation within a human body. In some instances, the electrodes 114/114′ connected to the LCP 100 may have an insulative portion that electrically isolates the electrodes 114/114′ from adjacent electrodes, the housing 120, and/or other parts of the LCP 100. In some cases, one or more of the electrodes 114/114′ may be provided on a tail (not shown) that extends away from the housing 120.

The processing module 110 can be configured to control the operation of the LCP 100. For example, the processing module 110 may be configured to receive electrical signals from the electrical sensing module 106 and/or the mechanical sensing module 108. Based on the received signals, the processing module 110 may determine, for example, abnormalities in the operation of the heart H. Based on any determined abnormalities, the processing module 110 may control the pulse generator module 104 to generate and deliver electrical stimulation in accordance with one or more therapies to treat the determined abnormalities. The processing module 110 may further receive information from the communication module 102. In some examples, the processing module 110 may use such received information to help determine whether an abnormality is occurring, determine a type of abnormality, and/or to take particular action in response to the information. The processing module 110 may additionally control the communication module 102 to send/receive information to/from other devices.

In some examples, the processing module 110 may include a pre-programmed chip, such as a very-large-scale integration (VLSI) chip and/or an application specific integrated circuit (ASIC). In such embodiments, the chip may be pre-programmed with control logic in order to control the operation of the LCP 100. By using a pre-programmed chip, the processing module 110 may use less power than other programmable circuits (e.g. general purpose programmable microprocessors) while still being able to maintain basic functionality, thereby potentially increasing the battery life of the LCP 100. In other examples, the processing module 110 may include a programmable microprocessor. Such a programmable microprocessor may allow a user to modify the control logic of the LCP 100 even after implantation, thereby allowing for greater flexibility of the LCP 100 than when using a pre-programmed ASIC. In some examples, the processing module 110 may further include a memory, and the processing module 110 may store information on and read information from the memory. In other examples, the LCP 100 may include a separate memory (not shown) that is in communication with the processing module 110, such that the processing module 110 may read and write information to and from the separate memory.

The battery 112 may provide power to the LCP 100 for its operations. In some examples, the battery 112 may be a non-rechargeable lithium-based battery. In other examples, a non-rechargeable battery may be made from other suitable materials, as desired. Because the LCP 100 is an implantable device, access to the LCP 100 may be limited after implantation. Accordingly, it is desirable to have sufficient battery capacity to deliver therapy over a period of treatment such as days, weeks, months, years or even decades. In some instances, the battery 112 may a rechargeable battery, which may help increase the useable lifespan of the LCP 100. A recharge circuit may receive power from a receiving coil of the LCP 100, and use the received power to recharge the rechargeable battery. In still other examples, the battery 112 may be some other type of power source, as desired.

To implant the LCP 100 inside a patient's body, an operator (e.g., a physician, clinician, etc.), may fix the LCP 100 to the cardiac tissue of the patient's heart. To facilitate fixation, the LCP 100 may include one or more anchors 116. The anchor 116 may include any one of a number of fixation or anchoring mechanisms. For example, the anchor 116 may include one or more pins, staples, threads, screws, helix, tines, and/or the like. In some examples, although not shown, the anchor 116 may include threads on its external surface that may run along at least a partial length of the anchor 116. The threads may provide friction between the cardiac tissue and the anchor to help fix the anchor 116 within the cardiac tissue. In other examples, the anchor 116 may include other structures such as barbs, spikes, or the like to facilitate engagement with the surrounding cardiac tissue.

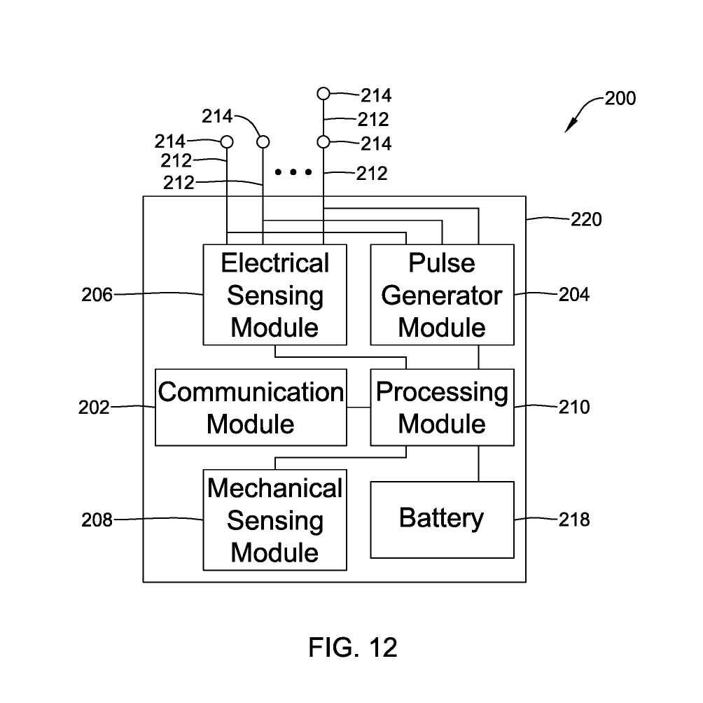

FIG. 12 depicts an example of another or second medical device (MD) 200, which may be used in conjunction with the LCP 100 (FIG. 11) in order to detect and/or treat cardiac abnormalities. In some cases, the MD 200 may be considered as an example of the IMD 12, the IMD 14, the IMD 16 (FIG. 1), the IMD 20 (FIG. 2), the IMD 50 (FIG. 5) or the LCP 60 (FIG. 6), and may for example represent an implantable cardioverter defibrillator (ICD) or a subcutaneous implantable cardioverter defibrillator (SICD). In the example shown, the MD 200 may include a communication module 202, a pulse generator module 204, an electrical sensing module 206, a mechanical sensing module 208, a processing module 210, and a battery 218. Each of these modules may be similar to the modules 102, 104, 106, 108, and 110 of LCP 100. Additionally, the battery 218 may be similar to the battery 112 of the LCP 100. In some examples, however, the MD 200 may have a larger volume within the housing 220. In such examples, the MD 200 may include a larger battery and/or a larger processing module 210 capable of handling more complex operations than the processing module 110 of the LCP 100.

While it is contemplated that the MD 200 may be another leadless device such as shown in FIG. 8, in some instances the MD 200 may include leads such as leads 212. The leads 212 may include electrical wires that conduct electrical signals between the electrodes 214 and one or more modules located within the housing 220. In some cases, the leads 212 may be connected to and extend away from the housing 220 of the MD 200. In some examples, the leads 212 are implanted on, within, or adjacent to a heart of a patient. The leads 212 may contain one or more electrodes 214 positioned at various locations on the leads 212, and in some cases at various distances from the housing 220. Some leads 212 may only include a single electrode 214, while other leads 212 may include multiple electrodes 214. Generally, the electrodes 214 are positioned on the leads 212 such that when the leads 212 are implanted within the patient, one or more of the electrodes 214 are positioned to perform a desired function. In some cases, the one or more of the electrodes 214 may be in contact with the patient's cardiac tissue. In some cases, the one or more of the electrodes 214 may be positioned subcutaneously and outside of the patient's heart. In some cases, the electrodes 214 may conduct intrinsically generated electrical signals to the leads 212, e.g. signals representative of intrinsic cardiac electrical activity. The leads 212 may, in turn, conduct the received electrical signals to one or more of the modules 202, 204, 206, and 208 of the MD 200. In some cases, the MD 200 may generate electrical stimulation signals, and the leads 212 may conduct the generated electrical stimulation signals to the electrodes 214. The electrodes 214 may then conduct the electrical signals and delivery the signals to the patient's heart (either directly or indirectly). In some cases, a transmit coil may be supported by the lead, such at a location along the length of the lead that is near the receive coil of a remote implantable medical device.

The mechanical sensing module 208, as with the mechanical sensing module 108, may contain or be electrically connected to one or more sensors, such as accelerometers, acoustic sensors, blood pressure sensors, heart sound sensors, blood-oxygen sensors, and/or other sensors which are configured to measure one or more mechanical/chemical parameters of the heart and/or patient. In some examples, one or more of the sensors may be located on the leads 212, but this is not required. In some examples, one or more of the sensors may be located in the housing 220.

While not required, in some examples, the MD 200 may be an implantable medical device. In such examples, the housing 220 of the MD 200 may be implanted in, for example, a transthoracic region of the patient. The housing 220 may generally include any of a number of known materials that are safe for implantation in a human body and may, when implanted, hermetically seal the various components of the MD 200 from fluids and tissues of the patient's body.

In some cases, the MD 200 may be an implantable cardiac pacemaker (ICP). In this example, the MD 200 may have one or more leads, for example the leads 212, which are implanted on or within the patient's heart. The one or more leads 212 may include one or more electrodes 214 that are in contact with cardiac tissue and/or blood of the patient's heart. The MD 200 may be configured to sense intrinsically generated cardiac electrical signals and determine, for example, one or more cardiac arrhythmias based on analysis of the sensed signals. The MD 200 may be configured to deliver CRT, ATP therapy, bradycardia therapy, and/or other therapy types via the leads 212 implanted within the heart. In some examples, the MD 200 may additionally be configured provide defibrillation therapy.

In some instances, the MD 200 may be an implantable cardioverter-defibrillator (ICD) with the ability to pace. In such examples, the MD 200 may include one or more leads implanted within a patient's heart. The MD 200 may also be configured to sense cardiac electrical signals, determine occurrences of tachyarrhythmias based on the sensed signals, and may be configured to deliver defibrillation therapy in response to determining an occurrence of a tachyarrhythmia. In other examples, the MD 200 may be a subcutaneous implantable cardioverter-defibrillator (S-ICD) with the ability to pace. In examples where the MD 200 is an S-ICD, one of the leads 212 may be a subcutaneously implanted lead. In some instances, the lead(s) may have one or more electrodes that are placed subcutaneously and outside of the chest cavity. In other examples, the lead(s) may have one or more electrodes that are placed inside of the chest cavity, such as just interior of the sternum but outside of the heart H.

In some examples, the MD 200 may not be an implantable medical device. Rather, the MD 200 may be a device external to the patient's body, and may include skin-electrodes that are placed on a patient's body. In such examples, the MD 200 may be able to sense surface electrical signals (e.g. cardiac electrical signals that are generated by the heart or electrical signals generated by a device implanted within a patient's body and conducted through the body to the skin). In such examples, the MD 200 may be configured to deliver various types of electrical stimulation therapy, including, for example, defibrillation therapy. In some cases, the MD 200 may be external to the patient's body may include a lead that extends transvenously into the heart. The lead may be used to sense and/or pace the heart. A transmit coil may be placed on the lead and adjacent to or inside of the heart.

It should be understood that this disclosure is, in many respects, only illustrative. Changes may be made in details, particularly in matters of shape, size, and arrangement of steps without exceeding the scope of the disclosure. This may include, to the extent that it is appropriate, the use of any of the features of one example embodiment being used in other embodiments.

What is claimed is:

1. An implantable medical device (IMD) comprising:
a housing;
a rechargeable battery disposed within the housing;
a battery recharging assembly disposed relative to the housing, the battery recharging assembly comprising:
an energy receiver disposed relative to the housing for capturing energy from an externally applied charging field;
a battery charging circuit disposed within the housing and operably coupled to the rechargeable battery for recharging the rechargeable battery;
a demodulator disposed within the housing and operably coupled to the energy receiver and the battery charging circuit, the demodulator demodulating the energy captured by the energy receiver and delivering demodulated energy to the battery charging circuit, the battery charging circuit using the demodulated energy to charge the rechargeable battery; and
a controller disposed within the housing and powered by the rechargeable battery, the controller configured to control operation of at least part of the IMD.

2. The IMD of claim 1, wherein the battery recharging assembly further comprises a switch that, when activated, is configured to prevent the battery charging circuit from recharging the rechargeable battery.

3. The IMD of claim 2, wherein the battery recharging assembly further comprises a switch that, when activated, is configured to switch off the energy receiver so that the energy receiver does not provide captured energy to the demodulator and thus the battery charging circuit.

4. The IMD of claim 3, wherein the energy receiver comprises a receiver coil having a first terminal and a second terminal, wherein the switch, when activated, effectively shorts the first terminal to the second terminal of the receiver coil.

5. The IMD of claim 1, wherein the energy receiver delivers a voltage to the demodulator, and the demodulator is configured to step up the voltage delivered by the energy receiver and provide the stepped up voltage to the battery charging circuit.

6. The IMD of claim 2, wherein the battery charging circuit is configured to provide a constant current to the rechargeable battery while a power level of the rechargeable battery remains below a first threshold.

7. The IMD of claim 6, wherein the battery charging circuit is configured to provide a constant voltage to the rechargeable battery once the power level of the rechargeable battery exceeds the first threshold but remains below a second threshold.

8. The IMD of claim 7, wherein the battery charging circuit is configured to activate the switch when the power level of the rechargeable battery reaches the second threshold.

9. The IMD of claim 1, wherein the energy receiver comprises an inductive energy receiver and/or an RF energy receiver.

10. The IMD of claim 1, wherein the energy receiver comprises a receiver coil supported on the housing.

11. The IMD of claim 10, wherein the receiver coil comprises a trace disposed on the housing.

12. The IMD of claim 1, further comprising a communications module that enables the controller to communicate with a remote device, and the controller is configured to receive charging instructions from the remote device via the communications module.

13. The IMD of claim 2, wherein the battery charging circuit is configured to monitor a remaining power level within the rechargeable battery, and to determine when to activate the switch based at least in part on remaining power level within the rechargeable battery.

14. The IMD of claim 1, wherein the energy receiver is configured to receive energy for recharging the rechargeable battery from another IMD.

15. The IMD of claim 1, wherein the IMD comprises a Leadless Cardiac Pacemaker (LCP) that includes a therapy module for delivering pacing therapy.

16. A leadless cardiac pacemaker (LCP) configured to sense cardiac signals from a patient's heart and to deliver pacing therapy to the patient's heart, the LCP comprising:
a housing;
a rechargeable battery disposed within the housing;
a battery recharging assembly disposed relative to the housing, the battery recharging assembly comprising:
an energy receiver disposed relative to the housing for capturing energy from an externally applied charging field;
a battery charging circuit disposed within the housing and operably coupled to the rechargeable battery for recharging the rechargeable battery;
a demodulator disposed within the housing and operably coupled to the energy receiver and the battery charging circuit, the demodulator demodulating the energy captured by the energy receiver and delivering demodulated energy to the battery charging circuit, the battery charging circuit using the demodulated energy to charge the rechargeable battery;
a switch that, when activated, is configured to prevent the battery charging circuit from recharging the rechargeable battery.
a pair of electrodes secured relative to the housing; and
a controller disposed within the housing and powered by the rechargeable battery, the controller is configured to sense cardiac signals and/or to deliver pacing therapy via the pair of electrodes.

17. The LCP of claim 16, wherein the battery charging circuit is configured to monitor a remaining power level within the rechargeable battery, and to determine when to activate the switch.

18. The LCP of claim 17, wherein:
the energy receiver delivers a voltage to the demodulator, and the demodulator is configured to step up the voltage delivered by the energy receiver and provide the stepped up voltage to the battery charging circuit;
the battery charging circuit is configured to provide a constant current to the rechargeable battery while a power level of the rechargeable battery remains below a first threshold;
the battery charging circuit is configured to provide a constant voltage to the rechargeable battery once the power level of the rechargeable battery exceeds the first threshold but remains below a second threshold; and
the battery charging circuit is configured to activate the switch when the power level of the rechargeable battery reaches the second threshold.

19. The LCP of claim 16, wherein the switch, when activated, is configured to switch off the energy receiver so that the energy receiver does not provide captured energy to the demodulator and thus the battery charging circuit.

20. A method of recharging an implantable device that has a rechargeable battery, the method comprising:
subjecting the implantable device to an energy field so that the implantable device is able to receive energy from the energy field;
allowing received energy to be used to recharge the rechargeable battery while a power level of the rechargeable battery is below a threshold; and
switching off received energy from being used to recharge the rechargeable battery when the power level of the rechargeable battery is at or above the threshold.
